(19) United States
(12) Patent Application Publication
(10) Pub. No.: US 2010/0249043 A1
(43) Pub. Date: Sep. 30, 2010

(54) COMPOSITIONS AND METHODS FOR PROMOTING HAIR GROWTH

(75) Inventor: Suan Lin, San Mateo, CA (US)

Correspondence Address: JOSEPH L. STRABALA, ESQ., ONE EMBARCADERO CENTER, SUITE 1020, SAN FRANCISCO, CA, 94111, US

(73) Assignee: LA CANADA VENTRURES, INC., Millsborough, CA (US)

(21) Appl. No.: 12/413,436

(22) Filed: Mar. 27, 2009

Related U.S. Application Data
(60) Provisional application No. 61/163,460, filed on Mar. 26, 2009.

(51) Int. Cl. A61K 38/02; A61P 17/00; A61Q 7/00

(52) U.S. Cl. 514/21

(57) ABSTRACT

The present invention relates to a composition for promoting hair growth and a method of use thereof. The composition comprises:

(a) 0.15 to 4.5 wt %, preferably 0.15 to 0.75 wt % of panthenyl ethyl ether,

(b) 0.15 to 4.5 wt %, preferably 0.15 to 0.75 wt % of inositol,

(c) 0.015 to 0.9 wt %, preferably 0.015 to 0.15 wt % of milk protein,

(d) 0.03 to 1.8 wt %, preferably 0.03 to 0.3 wt % of one or more sulfur-rich amino acids on the basis of the total weight of the said composition. The composition and method are capable of making hair much longer and thicker in relatively shorter time without any serious side effects.

### TECHNICAL FIELD OF THE INVENTION

The present invention relates to compositions and methods for promoting growth of eyelash and/or scalp hair. The compositions and methods are capable of making hair much longer and thicker in relatively shorter time without any serious side effects.

### BACKGROUND OF THE INVENTION

Hair loss is a common problem that is, for example, naturally occurring or chemically promoted by the use of certain therapeutic drugs designed to alleviate conditions such as cancer. Often such hair loss is accompanied by lack of hair re-growth that causes partial or full baldness.

The human hair growth cycle is essentially composed of three stages that repeat indefinitely in healthy follicles. The three known growth stages which have been identified are: (a) the anagen stage, during which the hair grows, (b) the catagen stage, during which the hair follicle prepares for the next phase, the telogen, and the previously growing hair is converted into a resting hair or club hair and (c) the telogen, which is the resting phase during which hair growth ceases completely. Using a mechanism, not yet fully understood, at some point at the end of the cycle, telogenic follicles commence forming new anagens, causing new hair to grow and existing hair to fall out. This growth cycle repeats itself unchanged during the life of the follicle and is essentially identical for vellous or terminal hair-producing follicles.

A desired for longer, thicker lashes by women is apparent by the abundance of cosmetic products that aim to temporarily alter the appearance of the user, i.e. mascara, lash extensions. Most of such products produce only temporary results, lasting a few hours to a few weeks. Eyelash transplants have been successful in producing longer lasting results, but the costs of transplantations are often very prohibitive to the general public.

Some products and compositions have been disclosed for enhancing hair growth, for example, an agent with aliphatic carboxylic acid for hair growing as described in U.S. Pat. No. 4,874,781; a hair tonic extract from dong chong xia cao as described in U.S. Pat. No. 4,769,231; an active ingredient Anageline® which is recommended in all hair treatment formulations to reduce hair loss and increase hair density; ViaPure-Sabal, a Saw Palmetto Extract which is used for reducing hair loss; Procapil™ used for hair strengthening and anti-hair loss treatment. However, scalp conditioners other than minoxidil have limited effect on scalp hair. Minoxidil cannot produce long lasting terminal hair, but only thin vellous hair.

Recently, synthetic prostaglandin analogues have been investigated for eyelash enhancement, see U.S. Pat. No. 7,351,404 “Method of Enhancing Hair Growth”. These compounds have abundant safety data for usage in intraocular in treatment of glaucoma, but lack rigorous studies on usage for topical application to improve appearance of eyelashes. However, lash conditioners with prostaglandin have the possible side effect of altering intraocular pressure, e.g. bimataprost, cause irreversible changes in iris color, irritation and vasodilation of conjunctiva causing erythema of the conjunctiva, and cannot be used in pregnancy or woman contemplating childbirth.

Therefore, there is a need for development of a hair growth composition and a method having superior hair growth-promoting effects without serious side effects.

### SUMMARY OF THE INVENTION

An object of the present invention is to provide a composition used for promoting the hair growth. The composition for promoting hair growth of the present invention comprises:

(a) 0.15 to 4.5 wt %, preferably 0.15 to 0.75 wt % of panthenyl ethyl ether,

(b) 0.15 to 4.5 wt %, preferably 0.15 to 0.75 wt % of inositol,

(c) 0.015 to 0.9 wt %, preferably 0.015 to 0.15 wt % of milk protein,

(d) 0.03 to 1.8 wt %, preferably 0.03 to 0.3 wt % of one or more sulfur-rich amino acids on the basis of the total weight of the said composition.

Another object of the present invention is to provide a hair conditioner comprising the composition mentioned above.

Still another object of the present invention is to provide a method for promoting hair growth by using the composition mentioned above. The hair includes scalp hair, eyelash and eyebrow.

The composition arid method are capable of making hair much longer and thicker in relative shorter time without any serious side effects.

### BRIEF DESCRIPTION OF THE DRAWINGS

FIG. 1 shows the comparison of the length of the eyelash before and after using the eyelash conditioner according to the present invention (the New MD Lash Factor).

### DETAILED DESCRIPTION OF THE INVENTION

Currently, Follicusan™ has become more attractive to the inventor, which is a compound mixture mainly containing amino acids. The recommended dosage of Follicusan™ is 3-5 wt %, and without reported skin irritation at 10 wt %. AGA study file indicates that Follicusan™ retards loss of androgen dependent alopecia due to stimulation of proliferation for dermal cells of hair follicles. However, so far, there is no data to demonstrate that Follicusan™ is able to result in the hair growth.

Panthenyl ethyl ether, inositol, milk protein (EU name: lactis proteinum) arid sulfur-rich amino acids are four biologically active signaling molecules found in Follicusan™ and their contents are 1-5 wt % of panthenyl ethyl ether, 1-5 wt % of inositol, 0.1-1 wt % of milk protein, 0.1-1 wt % of acetyl cysteine and 0.1-1 wt % of acetyl methionine respectively. Surprisingly, the inventor finds that combination of four ingredients mentioned above in higher level dosage would result in hair growth, especially growth of eyelash without any side effects.

In a first aspect, the present invention provides a composition for promoting hair growth, comprising:

(a) 0.15 to 4.5 wt %, preferably 0.15 to 0.75 wt % of panthenyl ethyl ether,

(b) 0.15 to 4.5 wt %, preferably 0.15 to 0.75 wt % of inositol,

(c) 0.015 to 0.9 wt %, preferably 0.015 to 0.15 wt % of milk protein,

(d) 0.03 to 1.8 wt %, preferably 0.03 to 0.3 wt % of one or more sulfur-rich amino acids on the basis of the total weight of the said composition.

Panthenyl ethyl ether (also named ethylpanthenol) is metabolized to pantothenic acid in the skin. Pantothenic acid is an essential element of coenzyme A, which plays a major role in metabolic processes. Ethylpanthenol is of high significance in hair cosmetics: it improves the condition of damaged hair, increases hair moisture, and protects the hair against damaging environmental influences and other stress factors.

Inositol has a metabolism-stimulating effect on the scalp and thus prevents hair damage.

Milk protein is a class of bioactive signaling molecules (cytokines) that are derived from milk. They vitalize the cells of the scalp including the hair follicles and stimulate protein biosynthesis.

The sulfur-rich amino acids, preferably N-acetyl cysteine, N-acetyl methionine or a mixture thereof counteract seborrhoic changes in the scalp, thus preventing formation of dandruff and accelerated greasing of the hair.

We test the compositions according to the present invention on scalp and eyelash at various ratios of the ingredients, and find that they all have the very positive effects on both areas. As for eyelashes, the results of our testing show that the compositions are able to produce faster and longer changes than prostaglandin.

In a preferable embodiment according to the present invention, the composition further comprises 0.001 to 0.2 wt %, preferably 0.0075 to 0.03 wt % of prostaglandin on the basis of the total weight of the composition. Prostaglandin is any member of a group of lipid compounds that are derived enzymatically from fatty acids and have important functions in animal body. Every prostaglandin contains 20 carbon atoms, including a 5-carbon ring. They are mediators and have a variety of strong physiological effects. Prostaglandin is found in virtually all tissues and organs. All nucleated cells except lymphocytes produce prostaglandin. We test the composition according to the present invention containing prostaglandin and find it work faster on scalp reducing the time of hair growth from 3 months to 1 month.

The composition according to the present invention further comprises 0.0.1 to 10 wt % of antioxidant on the basis of the total weight of the composition. Preferably, the antioxidant is L-ergothioneine. L-ergothioneine (LE) is a main chemical ingredient of Thiotaine® with the following chemical structure:

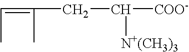

which is a unique, naturally occurring antioxidant that is abundant in most plants and animals, e.g. mushrooms, vegetables and grains. LE cannot be synthesized by human and therefore is available only from dietary source. The avidity by which dietary LE is assimilated by tissues, the specific and significant effects it has on cellular processes, and the degree to which it is conserved by cells suggest an important physiological role for this molecule. It has been shown that LE plays a dual role in both energy regulation and in protecting cells from oxidative damage. High concentrations of LE are found in a number of organ systems including liver, kidney, eye, seminal fluid and erythrocyte. LE is a powerful antioxidant that is stronger than CoQ10 or Idebenone.

The composition according to the present invention may comprise aqueous-alcoholic medium which comprises 10 to 30 wt % of denatured alcohol on the basis of the total weight of the aqueous-alcoholic medium.

The composition according to the present invention also further comprises hair/skin conditioner agent, such as panthenol, biothin, hydrolyzed glycosaminoglycan and proline; humectant, such as sorbitol, sodium lactate and sodium PCA; emollient, such as C₁₂₋₁₅ alkyl benzoate and caprylyl glycol; film former, such as acrylates/C₁₀₋₃₀ alkyl acrylate crosspolymer; buffering agent, such as sodium phosphate and sodium citrate; viscosity adjusting agent, such as sodium chloride; carrier/solvent other than aqueous-alcoholic medium; pH adjuster, such as triethanolamine and citric acid; thickening agent, such as xanthan gum; plant extract, such as grapeseed extract, rosemary extract, thyme extract, kudzu extract and horseschesnut extract; excipient; cosmetic base and/or gelling agent etc. For example, the composition the present invention may comprises 0.015 to 0.9 wt %, preferably 0.015 to 0.15 wt % of lactose; 0.015 to 0.99 wt %, preferably 0.015 to 0.165 wt % of sodium citrate and citric acid on the basis of the total weight of the composition.

In addition, the composition according to the present invention usually contains one or more preservatives selected from the group consisting of phenoxyethanol of about 0.075 to 0.5 wt %, methylparaben of about 0.015 to 0.1 wt %, sodium salt of dehydroacetic acid (Na-salt) of about 0.03 to 0.2 wt % based on the total weight of the composition.

In a preferable embodiment of the present invention, the composition comprises the following ingredients on the basis of the total weight of the said composition:

| | | |
|---|---|---|
| panthenyl ethyl ether | 0.15-4.5 | wt % |
| inositol | 0.15-4.5 | wt % |
| milk protein (lactis proteinium) | 0.015%-0.9 | wt % |
| lactose | 0.015%-0.9 | wt % |
| acetyl cysteine | 0.015%-0.9 | wt % |
| acetyl methionine | 0.015%-0.9 | wt % |
| citric acid | <0.095 | wt % |
| denatured alcohol | 1.5%-22.5 | wt % |

In a second aspect, the present invention provides a hair conditioner comprising the composition described above, preferably a scalp hair conditioner or eyelash conditioner.

In a third aspect, the present invention provides a method for promoting hair growth comprising administering to a subject the compositions described above. Specifically, the said method comprises contacting a subject with the composition described above, e.g. applying the composition described above to the roots of the hair daily with a fine brush. The hair includes scalp hair, eyelash and/or eyebrow.

The further uses and advantages of the present invention will be well explained by the following examples with reference to the accompanying drawings, which cannot be believed to limit the scope of the invention.

### EXAMPLE 1

An eyelash conditioner (the New MD Lash Factor) is prepared according to the following formulation (the percentage based on the total weight of the composition):

| | |
|---|---|
| panthenyl ethyl ether | 0.45% |
| inositol | 0.45% |
| milk protein (lactis proteinium) | 0.09% |
| lactose | 0.09% |
| acetyl cysteine | 0.09% |
| acetyl methionine | 0.09% |
| sodium citrate | 0.09% |
| citric acid | <0.015% |
| alcohol denat. | 10% |
| acrylates/C₁₀₋₃₀ alkylacrylate crosspolymer | 0.70% |
| xanthan Gum | 0.15% |
| C₁₂-C₁₅ alkyl benzoate | 0.50% |
| phenoxyethanol | 0.54% |
| caprylyl glycol | 0.43% |
| hydrolyzed glycosaminoglycan | 0.75% |
| triethanolamine | 0.52% |
| D-biotin | 0.10% |
| Panthenol | 0.50% |
| prodew 300 | 0.15% |
| alpha-bisabolol, natural | 0.10% |
| Thiotaine | 5% |
| water | q.s. |

The water-alcohol mixture is prepared with stirring, then added are the other components including the active proteins obtained from milk by fractionation.

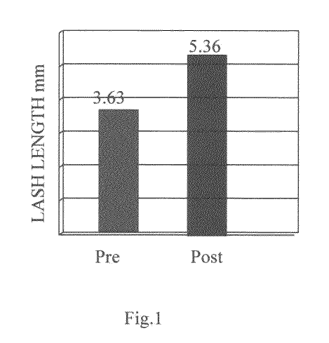

To assess the effectiveness of the eyelash conditioner (the New MD Lash Factor) to improve the appearance of eyelash including the enhanced length and thickness, 12 subjects are enrolled into the study. They are asked to apply a common lash conditioner on left eyelash and the New MD Lash Factor on right eyelash. The method of use is to apply the lash conditioners on the bed of the follicles of right eye upper eyelash nightly with a fine brush. Right eyelash measurements were taken in the medial, middle and lateral regions of both upper and lower lashes using micro calipers. The average of the measurements was then compared before and after 30 days of use. After one month of daily use, all the Subjects report enhanced eyelash thickness. The average increase in eyelash length on the right side is 1.73 mm. A 48% average increase in the length of the right eyelash is noted at end of 4 weeks as shown in FIG. 1, table 1 and 2. No irritation or side effects reported.

TABLE 1
the measurements of eyelash length before using the New
MD Lash Factor

Measurements before
the treatment

| | No. 1 | No. 2 | No. 3 | Mean |
|---|---|---|---|---|
| Subject No. | (mm) | (mm) | (mm) | (mm) |
| 1 | 3.5 | 4 | 3 | 3.917 |
| 2 | 2.5 | 3 | 2.5 | 3.583 |
| 3 | 2.5 | 2.5 | 2.5 | 2.875 |
| 4 | 3.5 | 3.5 | 2 | 3.208 |
| 5 | 3 | 3.5 | 3 | 3.533 |
| 6 | 3 | 3.5 | 3 | 3.417 |
| 7 | 3.5 | 3.5 | 2.4 | 3.400 |
| 8 | 2 | 2.5 | 2.5 | 3.208 |
| 9 | 1.5 | 2.5 | 1.5 | 2.708 |
| 10 | 2.9 | 3.3 | 2.3 | 3.800 |
| 11 | 2 | 3.5 | 3.5 | 4.750 |
| 12 | 3.5 | 4.5 | 4.5 | 5.167 |
| Mean value | | | | 3.631 |

TABLE 2
the measurements of eyelash length after using the New MD
Lash Factor

| | Post-treatment measurement | | | | | |
|---|---|---|---|---|---|---|
| | No. 4 | No. 5 | No. 6 | No. 7 | No. 8 | Mean |
| Subject No. | (mm) | (mm) | (mm) | (mm) | (mm) | (mm) |
| 1 | 6.5 | 5 | 4 | 5 | 5 | 4.917 |
| 2 | 7 | 6.5 | 7 | 5 | 5.25 | 5.792 |
| 3 | 6 | 6.5 | 5 | 4 | 4 | 4.833 |
| 4 | 6 | 5.75 | 4.5 | 5 | 5.5 | 4.958 |
| 5 | 7.5 | 6.5 | 4.5 | 5.5 | 6.5 | 5.833 |
| 6 | 4.5 | 5.5 | 4.6 | 4.5 | 4 | 4.517 |
| 7 | 7 | 7.75 | 6 | 4.5 | 4.5 | 5.708 |
| 8 | 4.5 | 5.5 | 4.5 | 4.5 | 4 | 4.333 |
| 9 | 4 | 5.5 | 4 | 3.5 | 4 | 4 |
| 10 | 6.2 | 7 | 5.1 | 3.5 | 4 | 4.983 |
| 11 | 9.5 | 12 | 8.5 | 3 | 6 | 7.333 |
| 12 | 9 | 10 | 7 | 5 | 6 | 7.167 |
| Mean value | | | | | | 5.365 |

In some subjects, the New MD Lash Factor results in longer growth than the existing product containing prostaglandin. For the New MD Lash Factor, the increase is noted approximately 80% at 4 weeks, while approximately 50% for the eyelash conditioner containing the prostaglandin.

### EXAMPLE 2

A scalp hair conditioner is prepared according to the following formulation (the percentage based on the total weight of the composition):

| | |
|---|---|
| panthenyl ethyl ether | 0.6% |
| inositol | 0.6% |
| milk protein (lactis proteinium) | 0.075% |
| lactose | 0.075% |
| acetyl cysteine | 0.075% |
| acetyl methionine | 0.075% |
| dechloroethylcloprostenolamide | 0.03% |
| sodium citrate | 0.075% |
| citric acid | <0.015% |
| SD Alcohol 40 | 0.03% |
| alcohol denat. | 5% |
| acrylates/C₁₀₋₃₀ alkylacrylate crosspolymer | 0.35% |
| C₁₂-C₁₅ alkyl benzoate | 0.50% |
| phenoxyethanol | 0.54% |
| caprylyl glycol | 0.43% |
| hydrolyzed glycosaminoglycan | 0.75% |
| D-biotin | 0.10% |
| panthenol | 0.50% |
| Cayman Active | 0.03% |
| triethanolamine | 0.52% |
| prodew 300 | 0.15% |
| alpha-bisabolol, natural | 0.10% |
| sodium chloride | 0.13% |
| Thiotaine | 3% |
| niacin | 0.05% |
| pyridoxine | 0.05% |
| retinyl palmitate | 0.05% |
| jojoba oil | 0.01% |
| water | q.s. |

The water-alcohol mixture is prepared with stirring, then added are the other components including the active proteins obtained from milk by fractionation except dechloroethylcloprostenolamide.

The subjects were asked to apply the scalp serum with various concentrations of prostaglandin (dechloroethylcloprostenolamide from 0 wt % to 0.03 wt %), and a placebo is applied on the separate portion of the scalp. Some subjects were followed with the scalp hair conditioner mention above or Saw Palmetto Oil (a peripheral testosterone inhibitor), and the other subjects do not. All the agents are applied directly to dry scalp after cleansing. Follow up are done monthly and photographs are taken.

The subjects treated by the scalp hair conditioner according to the present invention are noted to have faster response from 3 month down to 1 month comparing with the subjects treated by prostaglandin only. Average growth rate is 1-1.5 cm per month. Terminal hair was noted after 3 months. No significant difference noted with usage of Saw Palmetto Oil. The results show that the scalp hair conditioner according to the present invention containing prostaglandin expedites the effect on scalp to produce hair.

While the present invention has been described with reference to several preferred examples, it is contemplated that various alterations and modifications thereof will become apparent to those skilled in the art upon a reading of the preceding detailed description. It is therefore intended that the following appended claims be interpreted as including all such alterations and modifications falling within the true spirit and scope of this invention.

What is claimed is:

1. A composition for promoting hair growth, comprising:
(a) 0.15 to 4.5 wt %, preferably 0.15 to 0.75 wt % of panthenyl ethyl ether,
(b) 0.15 to 4.5 wt %, preferably 0.15 to 0.75 wt % of inositol,
(c) 0.015 to 0.9 wt %, preferably 0.015 to 0.15 wt % of milk protein,
(d) 0.03 to 1.8 wt %, preferably 0.03 to 0.3 wt % of one or more sulfur-rich amino acids on the basis of the total weight of the said composition.

2. The composition according to claim 1, wherein the sulfur-rich amino acids are acetyl cysteine, acetyl methionine or a mixture thereof.

3. The composition according to claim 1, further comprising 0.001 to 0.2 wt %, preferably 0.0075 to 0.03 wt % of prostaglandin on the basis of the total weight of the composition.

4. The composition according to claim 1, further comprising 0.01 to 10 wt % of antioxidant on the basis of the total weight of the composition.

5. The composition according to claim 4, wherein the antioxidant is L-ergothioneine.

6. The composition according to claim 1, further comprising aqueous-alcoholic medium that comprises 10 to 30 wt % of denatured alcohol on the basis of the total weight of the said aqueous-alcoholic medium.

7. The composition according to claim 1, further comprising 0.015 to 0.9 wt %, preferably 0.015 to 0.15 wt % of lactose on the basis of the total weight of the composition.

8. The composition according to claim 1, further composing 0.015 to 0.99 wt %, preferably 0.015 to 0.165 wt % of sodium citrate and citric acid on the basis of the total weight of the composition.

9. The composition according to claim 1, further comprising hair/skin conditioner agent, such as panthenol, biothin, hydrolyzed glycosaminoglycan and proline; humectant, such as sorbitol, sodium lactate and sodium PCA; emollient, such as C₁₂₋₁₅ alkyl benzoate and caprylyl glycol; film former, such as acrylates/C₁₀₋₃₀ alkyl acrylate crosspolymer; buffering agent, such as sodium phosphate and sodium citrate; viscosity adjusting agent, such as sodium chloride; carrier/solvent other than aqueous-alcoholic medium; pH adjuster, such as triethanolamine and citric acid; thickening agent, such as xanthan gum; plant extract, such as grapeseed extract, rosemary extract, thyme extract, kudzu extract and horseschesnut extract; excipient; cosmetic base and/or gelling agent etc.

10. A hair conditioner comprising the composition according to any of claims 1 to 10.

11. An eyelash conditioner comprising the composition according to any of claims 1 to 10.

12. A scalp hair conditioner comprising the composition according to claim 2.

13. A method for promoting hair growth, comprising administering to a subject the composition according to any of claims 1 to 10.

14. The method according to claim 13, comprising contacting a subject with the composition according to any of claims 1 to 10.

15. The method according to claim 13 or 14, wherein the hair is scalp hair, eye lash and/or eyebrow.
